(19) United States
(12) Patent Application Publication
(10) Pub. No.: US 2008/0114346 A1
(43) Pub. Date: May 15, 2008

(54) Cryosurgical Instrument

(75) Inventors: Alexander LEVIN, Binyamina (IL); Didier TOUBIA, Raanana (IL)

Correspondence Address: BRUCE E. LILLING;LILLING & LILLING PLLC, P.O. BOX 560, GOLDEN BRIDGE, NY, 10526, US

(73) Assignee: ARBEL MEDICAL LTD., Yokneam (IL)

(21) Appl. No.: 11/857,085

(22) Filed: Sep. 18, 2007

Related U.S. Application Data
(60) Provisional application No. 60/845,266, filed on Sep. 18, 2006.

(51) Int. Cl. A61B 18/02

(52) U.S. Cl. 606/23

(57) ABSTRACT

A cryosurgical probe (cryoprobe) for ablation of a superficial tissue. The cryoprobe operation is preferably based on combination of two freezing effects: a) heat transfer from a liquid, gaseous or aerosolized cryogen, which is delivered from openings in the cryoprobe tip immediately on the surface of the treated tissue, and b) heat transfer from needle(s) installed on the outer surface of the cryotip. This needle (or needles) is constructed from material with high thermal conductivity. In addition, the outer surface of the cryotip may be provided with spacers, which limit the depth of penetration of the needle(s) into the tissue.

### FIELD OF THE INVENTION

The present invention is related to the field of cryosurgical equipment, and, specifically, to cryosurgical instruments intended to ablate an unwanted superficial tissue.

### BACKGROUND OF THE INVENTION

In many cases, cryosurgical treatment of unwanted superficial tissue requires relatively deep freezing with respect to the effective diameter of the tissue. Application of cryoprobes, which are shaped as needles, can solve this problem. Some designs of such cryoprobes are proposed in Rabin (U.S. Pat. No. 6,786,902), Korpan (U.S. Pat. No. 6,565,556), Har-Shai (U.S. Pat. No. 6,503,246) and Makower (US Patent Publication No. 2005/0240147).

However, the cryoprobes in these patents and patent application describe complicated and expensive instruments, which, in addition, do not permit construction of a cryoprobe with a needle of a small diameter with a relatively high freezing capacity.

Therefore, there is a need to design a simple and reliable needle cryoprobe, which can penetrate into a tissue to be destroyed and effectively freeze this tissue.

### SUMMARY OF THE INVENTION

The background art does not teach or suggest a simple and reliable needle cryoprobe, which can penetrate into a tissue to be destroyed and effectively freeze this tissue.

The present invention overcomes these drawbacks of the background art by providing a cryoprobe which provides a combination of at least two freezing functions: a) heat transfer from a liquid, gaseous or aerosolized cryogen, which is delivered from openings in the cryoprobe tip preferably close to the surface of the treated tissue; and, b) heat transfer from needle(s) installed on the outer surface of the cryotip. This needle (or needles) is preferably constructed from material with high thermal conductivity. The needle(s) are preferably located on or in the surface of the tissue to be treated. This combination provides direct and indirect cooling of the tissue, providing synergistic treatment. The effect is further increased because the cooling material which is delivered from openings in the cryoprobe tip also cools the needles from the outside of the needles to the inside, while known in the art needles are cooled only from the inside of the needles to the outside.

In addition, the outer surface of the cryotip may optionally be provided with one or more spacing elements, which limit the depth of penetration of the needle(s) into the tissue. As shown in greater detail below, these spacing elements may optionally be separate from the needles (as spacers) or alternatively (or additionally) may optionally be part of the needles.

According to preferred embodiments, the cryoprobe preferably comprises a main lumen, the proximal end of this lumen being provided with an inlet connection for delivery of a cryogen in the form of gas, liquid or aerosol, and its distal end being sealed with a face plane member, which is provided with openings and outer needles installed on this face plane member. In addition, the outer surface of the face plane member can optionally be provided with spacers, which limit the depth of the needles' penetration into the treated tissue. This ensures flow of the cryogen, which emerges from the openings of the face plane member, preferably along the axes of the needles with effective heat transfer between the cryogen stream and the surface of the needles.

It is possible to deliver the cryogen into the internal space of the main lumen via a central feeding lumen with its distal end positioned in the immediate vicinity of the face plane member. This allows significant reduction of heat transfer between the cryogen and the internal wall of the main lumen itself.

Optionally, an additional external lumen is provided surrounding the main lumen. The gap between these lumens may optionally be provided with a suction mechanism for suction of the gaseous cryogen mixed with the surrounding air. This optional embodiment permits the cryoprobe of the present invention to be used for cryosurgical treatments in the internal cavities of a human body. In addition, this embodiment provides a solution for treatment of a specific area, for example, for treatment of a specific area of skin.

The outer surface of the main lumen can optionally be provided with a layer of thermal insulation.

In addition, if a central feeding lumen is provided, it is possible to place a thermo-insulating insert in the form of a tubular member between the main lumen and the central feeding lumen.

The needles and the spacers can optionally be designed as disposable elements. In this case, the proximal ends of the needles, the spacers and the face plane member are preferably provided with fasteners for fastening these needles and the spacer on the face plane member. There are a number of optional variants of joining the face plane member with the needles and spacers installed on it, and the main lumen. For example, the fasteners may optionally comprise, but are not limited to, threading, connectors of a bayonet type and others.

In order to diminish the time required for thawing the frozen tissue, it is possible to optionally deliver a gas at room temperature into the cryoprobe. Alternatively, delivery of the gas with sufficiently low temperature may optionally increase the required thawing time with an increase of ablation effect as a result of the freezing-thawing process.

The needles may optionally be constructed from materials that are known in the art, including but not limited to, metal with high thermal conductivity (silver, gold, stainless steel, bronze, alloys on the base of copper with nickel coating), or from composite material on the base of fibers with high thermal conductivity (carbon fibers). In addition, the needles may optionally be designed as closed or open pipes.

The needles may optionally be designed with changeable diameter along their height. In such a way, it is possible to combine the needle with the spacer, when the diameter of the needle at a specific height is diminished sharply. In addition, the needles may optionally be designed with gradually diminishment of the diameter in their distal direction.

In addition, the peripheral needles may optionally be shorter than the needles situated nearly the center. This difference in length facilitates penetration of the needles into a tissue, and, on the other hand, allows an optimal shape of an ice ball in the treated tissue to be obtained.

Optionally flexible fibers may be applied instead of needles. In this case these flexible fibers improve thermal contact between the cryotip and a treated tissue. The flexible elastic fibers are fabricated preferably from metal, for example, stainless steel. Without wishing to be limited by a single hypothesis, these flexible fibers perform three functions: they ensure good thermal contact with a tissue; they provide effective heat transfer to the cryogen stream and, on the other hand, they effectively conduct heat in their longitudinal direction.

According to preferred embodiments of the present invention, there is provided a cryoprobe constructed as a flexible catheter, in which the lumen(s) is(are) preferably constructed as flexible tubes. The material for the lumens may optionally comprise a corrugated material, including but not limited to, stainless steel (more preferably from about 10 microns to about 10 mm in thickness), Teflon or special polymers.

Hereinafter, the term “aerosol” includes but is not limited to mist (droplets of fluid in air), spray, atomized fluid particles, fluid suspended in a gas phase and/or small liquid drops in a gaseous medium.

### BRIEF DESCRIPTION OF THE DRAWINGS

FIG. 1A shows an axial cross-section of an exemplary, illustrative cryoprobe with an external thermal insulation and supply of a cryogen via the proximal end of a main lumen and FIG. 1B shows the distal end in more detail.

FIG. 2 is an axial cross-section of a cryoprobe with an external thermal insulation and supply of a cryogen via a central feeding lumen.

FIG. 3 is an axial cross-section of a cryoprobe with an internal thermal insulation and supply of a cryogen via a central feeding lumen.

FIG. 4 is an axial cross-section of a cryoprobe with an internal thermal insulation, supply of a cryogen via a central feeding lumen and suction of the mixture of the exhausted cryogen with the surrounding air.

FIG. 5 is an axial cross-section of the cryotip of the cryoprobe.

FIG. 6 shows an axial cross-section of a flexible cryocatheter with an external thermal insulation and supply of a cryogen via a central feeding lumen.

FIG. 7 shows an axial cross-section of an exemplary cryotip featuring flexible fibers rather than needles, which may optionally be implemented with any of the above cryoprobes.

### DESCRIPTION OF THE PREFERABLE EMBODIMENTS

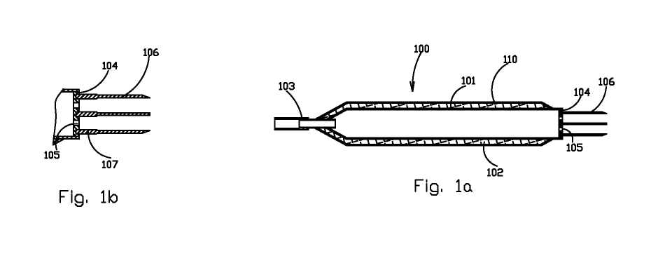

FIG. 1A shows an axial cross-section of an exemplary, illustrative cryoprobe with an external thermal insulation and supply of a cryogen via the proximal end of a main lumen, while FIG. 1B shows the distal end in more detail.

A cryoprobe 100 comprises: the main lumen 101 and an external thermal insulation 102 for insulating the cryogenic material within the main lumen 101. The external thermal insulation 102 may optionally comprise a vacuum, air, or any insulating material as is known in the art. Together the main lumen 101 and the external thermal insulation 102 preferably comprise an external shaft 110. An inlet connection 103 to the main lumen 101 is connected to a cryogen source (not shown). At the distal end of the main lumen 101, there is a face plane member 104, preferably with openings 105 and needles 106 installed on the outside surface of the face plane member 104. Cryogen emerges from openings 105 to a tissue to be treated, as well as to the cooling needles 106. The needles 106 are preferably inserted into the tissue to be treated (not shown).

In this embodiment, main lumen 101 preferably serves as the external lumen. There is no need for an internal lumen because the cryogen exits via openings 105 outwards.

FIG. 1B shows the distal end of cryoprobe 100 in more detail. As shown, one or more of needles 106 preferably features a thicker section 107, which acts as a spacing element to separate the needles 106 from the tissue being treated (not shown). The thicker section 107 is an example of a change or variation in the diameter of needles 106 along the length of needles 106 which may optionally be implemented for the spacing element. Preferably such a change features an increased diameter of needles 106 proximal to face plane member 104, as compared to a decreased diameter of needles distal to face plane member 104.

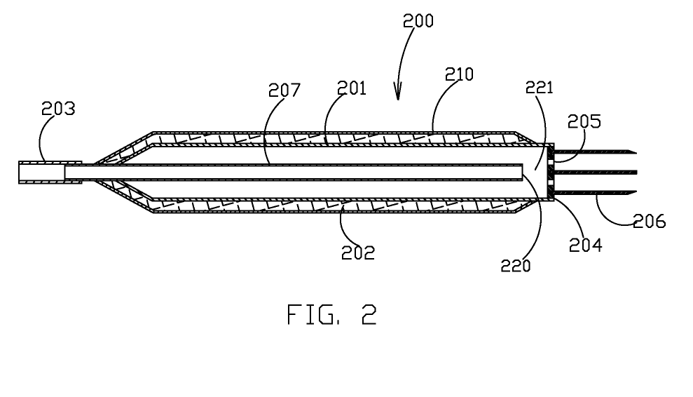

FIG. 2 demonstrates an axial cross-section of a cryoprobe with an external thermal insulation and supply of a cryogen via a central feeding lumen.

As shown in FIG. 2, a cryoprobe 200 comprises: the main lumen 201 and an external thermal insulation 202 for insulating the cryogenic material within the main lumen 201. Together the main lumen 201 and the external thermal insulation 202 preferably comprise an external shaft 210. In addition, within the main lumen 201, a central feeding lumen 207 is preferably provided for receiving the cryogen from inlet connection 203, which is connected to a cryogen source (not shown).

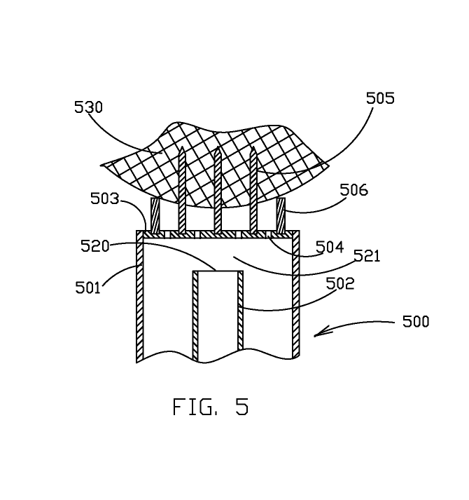

At the distal end of the main lumen 201, there is a face plane member 204, preferably with openings 205 and needles 206 installed on the outside surface of the face plane member 204. Cryogen emerges from the central lumen 207 through a central lumen opening 220, which may optionally be a plurality of central lumen openings 220 (not shown). Cryogen may then freely pass through an open space 221 and through a plurality of openings 205 to a tissue to be treated. Such cryogen also cools needles 206. Needles 206 are preferably inserted into the tissue to be treated (not shown, see FIG. 5).

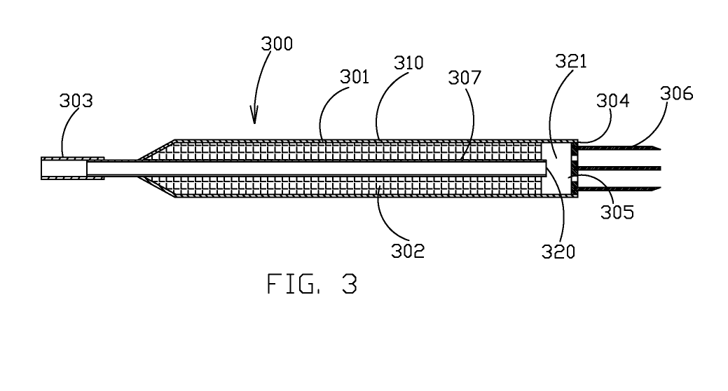

FIG. 3 shows an axial cross-section of a cryoprobe with an internal thermal insulation and supply of a cryogen via a central feeding lumen. A cryoprobe 300 comprises elements as for FIGS. 1 and 2; identical or similar elements are indicated with the same reference number as for FIG. 2, plus 100. However, in this embodiment, thermal insulation 302 is internal rather than being external as in FIG. 2, but may otherwise optionally comprise the same material.

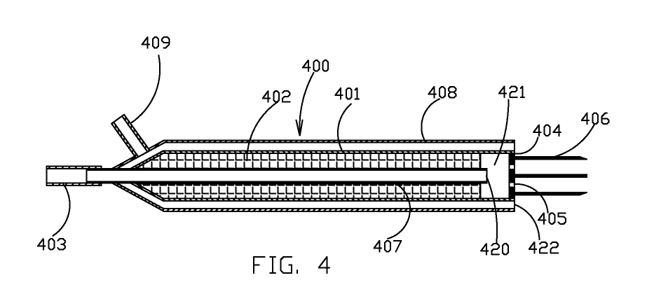

FIG. 4 is an axial cross-section of a cryoprobe with an internal thermal insulation, supplying a cryogen via a central feeding lumen and suctioning the mixture of the exhausted cryogen with the surrounding air.

A cryoprobe 400 comprises elements as in FIG. 3; identical or similar elements are indicated with the same reference number as for FIG. 3, plus 100. However, in this embodiment, rather than an external shaft 310, cryoprobe 400 features an external lumen 408 with a proximal outlet connection 409 to a suction device (not shown), for removing cryogen gas present in external lumen 408. The external lumen 408 is in suction communication with at least one opening 422 to remove the cryogen gas.

FIG. 5 shows an axial cross-section of the cryotip of the cryoprobe of FIG. 4.

A cryoprobe 500 is shown with only the distal sections 501 of the main lumen and 502 of the central feeding lumen for the purpose of illustration only. A face plane member 503 features openings 504 for cryogen to emerge and needles 505 to be cooled for treating a tissue (shown as reference number 530). Cryogen emerges from the central lumen 502 through a central lumen opening 520, which may optionally be a plurality of central lumen openings 520 (not shown). Cryogen may then freely pass through an open space 521 and through openings 504 to tissue 530. Such cryogen also cools needles 505. The face plane member 503 also preferably features a plurality of spacers 506, installed on the outer surface of the face plane member 503, to prevent direct contact of the openings 504 with the tissue to be treated 530.

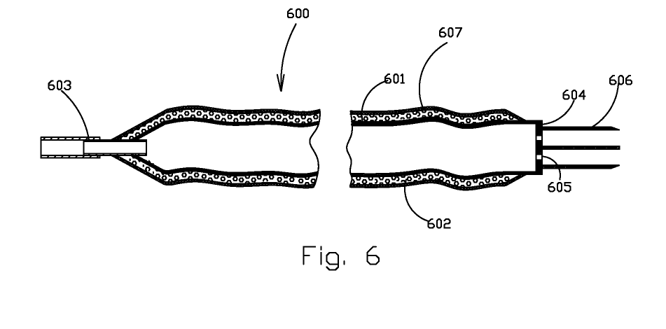

FIG. 6 shows an axial cross-section of a cryocatheter with an external thermal insulation and supply of a cryogen via a central feeding lumen. A cryoprobe 600 as shown is embodied as a cryocatheter, featuring a main lumen 601 and an external thermal insulation 602 for insulating the cryogenic material within the main lumen 601. The external thermal insulation 602 may optionally comprise a vacuum, air, or any insulating material as is known in the art. Together the main lumen 601 and the external thermal insulation 602 preferably comprise an external shaft 607; furthermore, main lumen 601 and external thermal insulation 602 are composed of one or more flexible materials as shown to be implemented as a cryocatheter. An inlet connection 603 to the main lumen 601 is connected to a cryogen source (not shown). At the distal end of the main lumen 601, there is a face plane member 604, preferably with openings 605 and needles 606 installed on the outside surface of the face plane member 604. Cryogen emerges from openings 605 to a tissue to be treated, as well as to the cooling needles 606. The needles 606 are preferably inserted into the tissue to be treated (not shown).

In this embodiment, main lumen 601 preferably serves as the external lumen. There is no need for an internal lumen because the cryogen exits via openings 605 outwards.

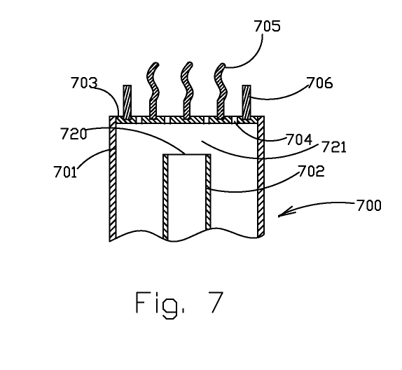

FIG. 7 shows an axial cross-section of an exemplary cryotip featuring flexible fibers rather than needles, which may optionally be implemented with any of the above cryoprobes. As shown, a cryocatheter 700 is shown with only the distal sections 701 of the main lumen and 702 of the central feeding lumen for the purpose of illustration only. A face plane member 703 features openings 704 for cryogen to emerge and a plurality of flexible fibers 705 to be cooled for treating a tissue (not shown). The flexible fibers 705 may optionally comprise metal or any other suitable material. Cryogen emerges from the central lumen 702 through a central lumen opening 720, which may optionally be a plurality of central lumen openings 720 (not shown). Cryogen may then freely pass through an open space 721 and through openings 704 to the tissue to be treated (not shown). Such cryogen also cools flexible fibers 705. The face plane member 703 also preferably features a plurality of spacers 706, installed on the outer surface of the face plane member 703, to prevent direct contact of the openings 704 with the tissue to be treated (not shown).

Any of the cryoprobes according to the present invention may optionally be used for a method of treatment of the skin, comprising: providing a cryogenic material to an interior portion of the cryoprobe, such that the material is able to leave through openings in the cryotip; placing at least one needle in contact with the area of skin to be treated; and permitting the cryomaterial to exit through openings in the cryotip. Optionally, the at least one needle may penetrate the skin. This method may optionally and preferably be used for treating a variety of skin conditions, including but not limited to, warts (including but not limited to plantar warts (verruca pedis) and genital warts), moles (nevi), pyogenic granulomas, dermatofibromas, dermoid cysts and other skin growths.

A cryoprobe according to the present invention which is adapted to become a cryocatheter (through the optional but preferred implementation of one or more lumen(s) with flexible material, as described above) may also optionally be used for treatment of an internal portion of the body, preferably through a method comprising inserting the cryocatheter into the body (optionally through an opening made for this purpose) and treating the tissue to be treated as described above.

Persons skilled in the art will appreciate that the present invention is not limited to what has been particularly shown and described hereinabove. Rather the scope of the present invention is defined by the appended claims and includes both combinations and sub combinations of the various features described hereinabove as well as variations and modifications thereof, which would occur to persons skilled in the art upon reading the foregoing description.

We claim:

1. A cryosurgical probe for treatment of tissues comprising:
a lumen provided with inlet means at a proximal end for delivery of cryogen into it, and having a distal end;
a face plane member provided with at least one opening and being positioned at and sealing the distal end of said lumen;
at least one needle installed on an outer side of said face plane member; and
at least one spacing element installed on the outer side of said face plane member to prevent direct contact of said at least one openings with the tissue.

2. The cryosurgical probe for treatment of tissues as claimed in claim 1, further comprising external thermal insulation for said lumen.

3. The cryosurgical probe for treatment of tissues as claimed in claim 1, further comprising a central feeding lumen situated in an internal space of the lumen, said central feeding lumen supplying the cryogen to an internal surface of the face plane member.

4. The cryosurgical probe for treatment of tissues as claimed in claim 1, wherein the cryogen is a liquid cryogen.

5. The cryosurgical probe for treatment of tissues as claimed in claim 1, wherein the cryogen is a gaseous cryogen.

6. The cryosurgical probe for treatment of tissues as claimed in claim 1, wherein the cryogen is a cryogenic aerosol.

7. The cryosurgical probe for treatment of tissues as claimed in claim 3, further comprising an internal thermal insulation situated between the lumen and the central feeding lumen.

8. The cryosurgical probe for treatment of tissues as claimed in claim 1, wherein said spacing element comprises a variable diameter of at least one of the needles to prevent direct contact of said at least one opening with the tissue.

9. The cryosurgical probe for treatment of tissues as claimed in claim 1, wherein said spacing element comprises at least one spacer.

10. The cryosurgical probe for treatment of tissues as claimed in claim 1, further comprising an external lumen around the lumen and a proximal outlet connection communicating with said external lumen, wherein cryogen in said external lumen is exhausted through said proximal outlet connection.

11. The cryosurgical probe for treatment of tissues as claimed in claim 1, wherein the needles comprise flexible fibers.

12. The cryosurgical probe for treatment of tissues as claimed in claim 11, wherein the flexible fibers are fabricated from metal.

13. The cryosurgical probe for treatment of tissues as claimed in claim 1, wherein said lumen is constructed of a flexible material to form a cryocatheter.
